(19) United States
(12) Patent Application Publication
(10) Pub. No.: US 2020/0200760 A1
(43) Pub. Date: Jun. 25, 2020

(54) COMPOSITIONS COMPRISING LIGANDS TO RHOB PROTEIN AND THE USES THEREOF

(71) Applicant: Lankenau Institute of Medical Research, Wynnewood, PA (US)

(72) Inventors: Lisa Laury-Kleintop, Chesterbrook, PA (US); George C. Prendergast, Penn Valley, PA (US)

(21) Appl. No.: 16/613,299

(22) PCT Filed: May 15, 2018
(86) PCT No.: PCT/US2018/032797

§ 371 Date: Nov. 13, 2019

Related U.S. Application Data
(60) Provisional application No. 62/506,798, filed on May 16, 2017.

(51) Int. Cl. G01N 33/58; G01N 33/539; G01N 33/68; G01N 33/577; G01N 33/543; C07K 17/14; C07K 17/02

(52) U.S. Cl. CPC G01N 33/582; G01N 33/539; G01N 33/6848; G01N 33/577; G01N 2800/52; C07K 17/14; C07K 17/02; G01N 2800/347; G01N 2800/368; G01N 33/543

(57) ABSTRACT

Methods and compositions are provided for diagnosing of autosomal-dominant polycystic kidney disease (ADPKD), chronic kidney diseases, kidney dysfunction, and preeclampsia in a subject, preferably in a urine sample of a human subject. The methods and compositions enable the detection or measurement in the sample or from a protein profile generated from the sample, of RhoB protein or peptide fragments thereof. Comparing the protein level(s) of the RhoB protein or peptide fragments thereof in the subject's sample with the level of the same protein or peptide(s) in a reference standard, permits the determination of a diagnosis of ADPKD and other said diseases, or the identification of a risk of developing ADPKD and other said diseases, or enables the monitoring of the status of progression or remission of ADPKD and other said diseases in the subject.

### CROSS-REFERENCE TO RELATED APPLICATIONS

This application claims the benefit of priority of U.S. provisional patent application No. 62/506,798, filed on May 16, 2017. The disclosure of this provisional application is incorporated by reference herein.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “MLH104PCT_ST25.txt”, prepared May 15, 2018 and is of 30 kB.

### BACKGROUND OF THE INVENTION

Chronic kidney disease (CKD, chronic renal disease) is an internationally recognized public health problem affecting 5-10% of the world population and the cause of 956,000 deaths globally in 2013, up from 409,000 in 1990. It presents as a progressive loss in kidney function over a period of months or years with various causes. Currently, renal function decline evaluated via blood and urine tests is utilized for diagnosing CKD. For example, creatinine, which is a breakdown product of muscle metabolism, can be measured in blood samples. Higher levels of creatinine indicate a lower glomerular filtration rate (GFR) and a decreased capability of the kidneys to excrete waste products. However, creatinine levels may be normal in the early stages of CKD, and the condition is discovered when a urinalysis test indicates the kidney is excreting protein or red blood cells into the urine.

Early diagnosis and treatment of the underlying cause and/or the institution of secondary preventive measures are imperative in patients with chronic kidney disease (CKD). One of the causes of CKD is autosomal dominant polycystic kidney disease (ADPKD, autosomal dominant PKD or adult-onset PKD), which is the most prevalent, potentially lethal, monogenic human disorder. Over 50% of patients with ADPKD eventually develop end stage kidney disease and require dialysis or kidney transplantation. ADPKD is estimated to affect at least 1 in every 1000 individuals worldwide, making this disease the most common inherited kidney disorder with a diagnosed prevalence of 1:2000 and incidence of 1:3000-1:8000 in a global scale.

Usually, the diagnosis of ADPKD is initially performed by renal imaging using ultrasound, CT scan, or MRI, which are costly procedures requiring special medical instruments and well-trained medical staff. Molecular genetic testing by linkage analysis or direct mutation screening is clinically available; however, genetic heterogeneity is a significant complication in molecular genetic testing. The large size and complexity of two genes associated with ADPKD, PKD1 and PKD2, as well as marked allelic heterogeneity, present obstacles to molecular testing by direct DNA analysis. The sensitivity of testing is nearly 100% for all patients with ADPKD who are 30 years of age or older and for younger patients with PKD1 mutations; these criteria are only 67% sensitive for patients with PKD2 mutations who are younger than 30 years of age.

Additionally, pre-eclampsia (PE) affects 2-8% of pregnancies worldwide. As a hypertensive disorder of pregnancy, pre-eclampsia is one of the most common causes of death due to pregnancy. It is a disorder characterized by the onset of high blood pressure and often a significant amount of proteins in the urine. In severe disease, there may be red blood cell breakdown, a low blood platelet count, impaired liver function, kidney dysfunction, swelling, shortness of breath due to fluid in the lungs, or visual disturbances. Pre-eclampsia increases the risk of poor outcomes for both the mother and the baby. If left untreated, it may result in seizures at which point it is known as eclampsia.

Pre-eclampsia is currently diagnosed when a pregnant woman develops elevated systolic/diastolic blood pressure, more than 300 mg of proteinuria in urine sample, a SPOT urinary protein to creatinine ratio at 0.3 or more, or a urine dipstick reading of 1+ or greater. There have been many assessments of tests aimed at predicting pre-eclampsia, though no single biomarker is likely to be sufficiently predictive of the disorder. Predictive tests that have been assessed include those related to placental perfusion, vascular resistance, kidney dysfunction, endothelial dysfunction, and oxidative stress. Studies have demonstrated that detecting podocytes in the urine may serve as an early marker of, and diagnostic test for, pre-eclampsia (CRAICI et al. 2013).

There remains a need in the art for a simpler, less invasive and more accurate diagnostic or predictive method for CKD, ADPKD or pre-eclampsia.

### SUMMARY OF THE INVENTION

In one aspect, a diagnostic reagent comprises a Ligand which is covalently linked to a detectable label or immobilized on an immobilization substrate and which is capable of specifically complexing with, binding to, identifying or quantitatively detecting a Target within a RhoB protein.

In another aspect, a diagnostic kit comprises a Ligand which is capable of specifically complexing with, binding to, identifying or quantitatively detecting a Target within a RhoB protein; and a Coating Peptide which is RhoB protein or a fragment thereof, associated with a detectable label or immobilized on an immobilization substrate and capable of complexing with, binding to the Ligand, is provided.

Another diagnostic kit comprising a Ligand immobilized on an immobilization substrate and capable of specifically complexing with, binding to, identifying or quantitatively detecting a Target within a RhoB protein is also provided.

In another aspect, a method is provided for diagnosing, detecting or monitoring the progress of a disease in a subject. The method comprises: contacting a sample obtained from a subject with a composition comprising: a Ligand which is capable of specifically complexing with, binding to, identifying or quantitatively detecting a Target within a RhoB protein, and a Coating Peptide which is RhoB protein or a fragment thereof, associated with a detectable label or immobilized on an immobilization substrate and capable of complexing with, binding to the Ligand; detecting the presence or measuring the levels of one or more Targets; and comparing the Target level(s) in the subject's sample with the level in a reference standard. The presence, absence, appearance, disappearance, significant increase or significant decrease in Target level(s) in the subject's sample compared to that in the reference standard indicates a diagnosis, risk, progression or remission of the disease in the subject.

In still another aspect, a method is also provided for diagnosing, detecting or monitoring the progress of a disease in a subject. This method comprises: performing co-immunoprecipitation (Co-IP), affinity chromatography and other pull-down assays via contacting a sample obtained from a subject with a composition comprising a Ligand capable of specifically complexing with, binding to, identifying or quantitatively detecting a Target within a RhoB protein to deplete non-Target molecule in the sample or to enrich the Target(s); performing mass spectrometry, gel electrophoresis, or other protein quantitation assays to identify the pulled-down Target(s) or to measure the Target level(s); comparing the Target level(s) in the subject's sample with the level(s) in a reference standard; wherein an the presence, absence, appearance, disappearance, significant increase or significant decrease in Target level(s) in the subject's sample compared to that in the reference standard indicates a diagnosis, risk, progression or remission of the disease in the subject.

In a further aspect, the methods described herein are for early diagnosis of disease, determining the best clinical treatment, monitoring relapse after initial diagnosis and treatment, or predicting clinical outcome.

Other aspects and advantages of these methods and compositions are described further in the following detailed description.

### BRIEF DESCRIPTION OF THE FIGURES

FIG. 1 provides results of a competitive ELISA identifying amino acids recognized by a RhoB antibody 7F7. Briefly, each well in an ELISA plate was coated with a Coating Peptide with an amino acid sequence of Acetyl-RTDDGRAMAVRIQAYDYLE-Amidyl (SEQ ID NO: 4). After blocking, competing peptides as indicated below as well as in FIG. 1 (circles, RhoB Peptide 1 with sequence of RTDDGRAMAVRIQAYDYLE, SEQ ID NO: 4; squares, RhoB Peptide 4 with sequence of AVRIQAYDYLE, SEQ ID NO: 7; triangles, RhoB Peptide 2 with sequence of DDGRAMAVRIQAY, SEQ ID NO: 5; and inverted triangles, RhoB Peptide 3 with sequence of RTDDGRAMAVRIQ, SEQ ID NO: 6) were incubated at various concentrations in the presence of the tested RhoB antibody 7F7. Horseradish peroxidase enzyme (HRP) conjugated secondary antibody recognizing 7F7 was applied to each well after appropriate washes. Chromogenic enzyme substrates were provided to produce a quantitative signal measured by a plate reader. A decline in absorbance upon increasing concentrations of the competing peptide indicates a successful binding between the tested antibody (7F7) and said competing peptide while a sustained absorbance indicates no such binding.

FIG. 2 provides results of a competitive ELISA showing that a RhoB antibody 7F7 recognizes the RhoB peptides, IQAYDYLECSAK, SEQ ID NO: 8 and IEAYDYLECSAK, SEQ ID NO: 9. Briefly, each well on an ELISA plate was coated with a Coating Peptide with an amino acid sequence of Acetyl-RTDDGRAMAVRIQAYDYLE-Amidyl, SEQ ID NO: 4. After blocking, the competing peptides as indicated below as well as in FIG. 2 (circles, RhoB peptide 1 with sequence of RTDDGRAMAVRIQAYDYLE, SEQ ID NO: 4; squares, RhoB Peptide 4 with sequence of AVRIQAYDYLE, SEQ ID NO: 7; triangles, CKD RhoB Peptide 1 with sequence of IQAYDYLECSAK, SEQ ID NO: 8; and inverted triangles, CKD RhoB Peptide 2 with sequence of IEAYDYLECSAK, SEQ ID NO: 9) were incubated at various concentrations in the presences of the tested RhoB antibody 7F7. Horseradish peroxidase enzyme (HRP) conjugated secondary antibody recognizing 7F7 was applied to each well after appropriate washes. Chromogenic enzyme substrates were provided to produce a quantitative signal measured by a plate reader. A decline in absorbance upon increasing concentrations of the competing peptide indicates a successful binding between the tested antibody (7F7) and said competing peptide while a sustained absorbance indicates no such binding.

### DETAILED DESCRIPTION OF THE INVENTION

Methods and compositions are provided that enable diagnosis of autosomal-dominant polycystic kidney disease (ADPKD), chronic kidney diseases, kidney dysfunction, and pre-eclampsia in a subject, as described herein. In one embodiment, methods and compositions are provided herein to enable the detection or measurement in a urine sample of a human subject or from a protein profile generated from the sample, of RhoB protein or peptide fragments thereof. The method involves comparing the protein level(s) of the RhoB protein or peptide fragments thereof in the subject's sample with the level of the same protein or peptide(s) in a reference standard. Such a method permits the determination of a diagnosis of ADPKD or other said diseases, or the identification of a risk of developing ADPKD or other said diseases, or enables the monitoring of the status of progression or remission of ADPKD or other said diseases in the subject.

The presence or functions of RhoB or fragment(s) thereof have been linked to various diseases, such as ADPKD (KONVALINKA et al. 2013, KONVALINKA et al. 2016), CKD (KONVALINKA et al. 2013, KONVALINKA et al. 2016, ZYNDA et al. 2016) and Pre-eclampsia (HAN et al. 2016). As described in the Examples, the inventors exhibited that a RhoB antibody named as 7F7 identifies the fragments of RhoB protein which have been shown to be present in the urine samples at significantly different levels among patients with CKD, those with ADPKD and healthy controls (KONVALINKA et al. 2016). These results demonstrated that the RhoB antibody 7F7 can serve as a diagnostic reagent and provide a method for diagnosing ADPKD and CKD.

### I. Definitions

Previous professional guidelines (ABBOUD et al. 2012) classified the severity of chronic kidney disease (CKD) in five stages based on glomerular filtration rate (GFR), with stage 1 being the mildest and usually causing few symptoms and stage 5 being a severe illness with poor life expectancy if untreated. The five stages are listed as follows: Stage 1 with normal or high GFR (GFR>90 mL/min); Stage 2 Mild CKD (GFR=60-89 mL/min); Stage 3A Moderate CKD (GFR=45-59 mL/min); Stage 3B Moderate CKD (GFR=30-44 mL/min); Stage 4 Severe CKD (GFR=15-29 mL/min); and Stage 5 End Stage CKD (GFR<15 mL/min).

The term “Ligand” refers to a molecule that is capable of specifically complexing with, binding to, identifying or quantitatively detecting a molecule. In one embodiment, the molecule is a protein or a peptide. In one embodiment, the Ligand is a molecule that is capable of specifically complexing with, binding to, identifying or quantitatively detecting a Target as described herein. In another embodiment, the Ligand is a molecule that is capable of specifically complexing with, binding to, identifying or quantitatively detecting another Ligand. Said Ligands can be selected from an antibody, an antibody mimic, an antibody equivalent, or a fragment thereof that binds to or complexes with a protein or a peptide.

As used herein, an “antibody” is a monoclonal antibody, a synthetic antibody, a recombinant antibody, a chimeric antibody, a humanized antibody, a human antibody, a CDR-grafted antibody, a multispecific binding construct that can bind two or more epitopes, a dual specific antibody, a bi-specific antibody, a multi-specific antibody, an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, an Fc construct, a monovalent or bivalent construct from which domains non-essential to monoclonal antibody function have been removed, a single-chain molecule containing one VL (variable region of light chain), one VH (variable region of heavy chain) antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, or any recombinant versions thereof. Methods for producing such antibodies are well-known in the art. Indeed, commercial vectors for certain antibody and antibody fragment constructs are available.

As used herein, an “antibody mimic” or an “antibody equivalent” refers to affibodies (LÖFBLOM et al. 2010), i.e., a class of engineered affinity proteins, generally small (˜6.5 kDa) single domain proteins that can be isolated for high affinity and specificity to any given target, aptamers (JAYASENA 1999), polypeptide molecules that bind to a specific target, an affilin (EBERSBACH et al. 2007), an affitin (MOURATOU et al. 2015), an affimer (JOHNSON et al. 2012), an alphabody (DESMET et al. 2014), an anticalin (SKERRA 2008), an avimer (SILVERMAN et al. 2005), a DARPin (STUMPP et al. 2008), a Fynomer (GRABULOVSKI et al. 2007), a Kunitz domain peptide (NIXON et al. 2006), a monobody (KOIDE et al. 2007), darpins (designed ankyrin repeat proteins; (KAWE et al. 2006)), peptabodies (TERSKIKH et al. 1997) and others known in the art.

As used herein, a “label” or “detectable label” is a chemical or biochemical moiety useful in association with the Ligand of the Target or the Coating Peptide, that alone or in concert with other components enable the detection of a Target via providing a detectable signal. In one embodiment, a detectable label or component does not naturally occur in association with the Ligand. Such labels or components include, without limitation, gold, colloidal gold, colored particles, colored latex beads, carbon nanoparticles, selenium nanoparticles, silver nanoparticles, quantum dots, up converting phosphors, organic fluorophores, textile dyes, colloidal carbon, liposomes, fluorescent agents, chemiluminescent agents, chromogenic agents, quenching agents, radionucleotides, enzymes, enzymatic substrates, cofactors, inhibitors, radioactive isotopes, magnetic particles, polypeptide tag, cleavage tag, and other moieties known in the art. In certain embodiment, the “labels” or “detectable labels” are covalently or non-covalently associated with the Ligand or the Coating Peptide. In a further embodiment, the label comprises a molecule which is capable of specifically complexing with, binding to, identifying or quantitatively detecting the Ligand; and which is associated with a chemical or biochemical moiety that alone or in concert with other components enable the detection of the Ligand and/or a Target via providing a detectable signal as described herein. Such labels are capable of generating a measurable signal alone, e.g., radioactivity, or in association with another component, e.g., an enzymatic signal in the presence of an enzymatic substrate. Methods of attaching the labels to the antigens are conventional.

In one embodiment, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as an enzymatic substrate) releases peroxide as a product that in the presence of peroxidase and a hydrogen donor such as tetramethyl benzidine (TMB) produces an oxidized TMB that is seen as a blue colour. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods and compositions described herein are detectable by other means, e.g., colored latex microparticles (Bangs Laboratories, Indiana) in which a dye is embedded may be used in place of enzymes to provide a visual signal indicative of the presence of the resulting Target-Ligand complex. Still other labels include fluorescent compounds, radioactive compounds or elements. Preferably, a Ligand is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon method.

A “polypeptide tag” is generally a short amino acid sequence incorporated into a heterologous polypeptide sequence that facilitates the identification and/or purification of the polypeptide sequence to which it is attached. For example, a useful polypeptide tag is a Myc tag, a FLAG Tag, a NE Tag, a HA-Tag, a His or poly-His Tag. Other suitable tags include without limitation, a tobacco etch virus (TEV) protease recognition site. Still other suitable polypeptide tags may be used in the compositions and methods described herein.

By “cleavage tag” is generally meant a short amino acid sequence incorporated into a heterologous polypeptide sequence that allows the polypeptide to be cleaved at that site by an enzymatic or other mechanism. In one example, a useful cleavage tag is the 3C PreScission protease or PSP cleavage tag. In another example, a tag is an EKT (Enterokinase) cleavage tag. In another embodiment, a tag is a FXa (Factor Xa) cleavage tag. In still another embodiment, a tag is a TEV (tobacco echovirus) cleavage tag. In still another example, a tag is a thrombin cleavage tag. Still other suitable cleavage tags may be used in the compositions and methods described herein.

As used herein, the term “enzyme substrate” or “enzymatic substrate” refers to a molecule upon which an enzyme acts.

As used herein, the term “immobilization substrate” refers to any material on which the Ligand or the Coating Peptide can be immobilized and includes, without limitation, materials such as silicon, glass, PDMS, plastic, metal, gold, colloidal gold, potentiometric crystal, quartz crystal balance (QCM), quartz, nanoparticle, colored particles, colored latex beads, carbon nanoparticles, selenium nanoparticles, silver nanoparticles, quantum dots, colloidal carbon, liposomes, nanoporous alumina, polydopamine, silicon, hydrogel, sol-gel, polycarbonate (PC) membrane, nitrocellulose (NC) membrane, PVDF (polyvinylidene fluoride) membrane, cellulose membrane, ferromagnetic materials and polymers in embodiments such as a chromatography column, a multi-well plate, a microarray, a microfluidics card, a chip, a piezoelectric chip, a bead, a magnetic particle, a lateral flow device, a piezoelectric device, a microcantilever, a biosensor, a nanowire, a lab-on-a-chip, or a chamber. In one embodiment, immobilization can be achieved via physisorption (intermolecular forces, such as electrostatic, hydrophobic, van der Waals, hydrogen bonding interactions, or combination of those), bioaffinity interaction, covalent bond, use of spacer to the selected immobilization substrate. Methods and techniques of immobilization are known in the art. See, e.g. (KIM et al. 2013).

As used herein, “disease”, “disorder” and “condition” are used interchangeably, to indicate an abnormal state in a subject. In one embodiment, the disease is autosomal-dominant polycystic kidney disease (ADPKD), chronic kidney diseases (CKD), kidney dysfunction, or pre-eclampsia. In one embodiment, the disease is ADPKD. In another embodiment, the disease is CKD which is not caused by ADPKD. In still another embodiment, the disease is CKD caused by high blood pressure, type 1 diabetes, type 2 diabetes, glomerulonephritis, Interstitial nephritis, Polycystic kidney disease, Prolonged obstruction of the urinary tract, from conditions such as enlarged prostate, kidney stones and some cancers, Vesicoureteral reflux, or Recurrent kidney infection (pyelonephritis).

“Patient” or “subject” as used herein means a male or female mammalian animal, including a human, a veterinary or farm animal, a domestic animal or pet, and animals normally used for clinical research. In one embodiment, the subject of these methods and compositions is a human. In one embodiment, the subject of these methods and compositions is a male or female human.

“Sample” as used herein means any biological fluid or tissue of a subject. The most suitable samples for use in the methods and with the compositions are urine samples. In another embodiment, the samples are blood samples, including serum, plasma, whole blood, and peripheral blood. It is also anticipated that other biological fluids, such as saliva, vaginal or cervical secretions, amniotic fluid, cerebrospinal fluid (CSF) and placental fluid may be used similarly. Such samples may further be diluted with saline, buffer or a physiologically acceptable diluent. Alternatively, such samples are concentrated by conventional means. The sample may be provided at any time that is considered biologically relevant to the physician or healthcare provider. In one embodiment, the subject's sample has been provided at a time before, during or after any of the following procedures, such as diagnosis, therapeutic treatment, surgical treatment, or an adjustment of a therapy.

“Reference standard” as used herein refers to the source of the reference Target levels. The “reference standard” is preferably provided by using the same technique as is used for measurement of the subject's Target levels in the reference subject or population, to avoid any error in standardization. The reference standard is, alternatively, a numerical value, a predetermined cutpoint, a mean, an average, a numerical mean or range of numerical means, a numerical pattern, a ratio, a graphical pattern or a protein abundance profile or protein level profile derived from the same Target or Targets in a reference subject or reference population. In an embodiment, the reference standard can be an expression level of one or more Target(s) or a ratio of expression level between two different Targets.

“Reference subject” or “Reference Population” defines the source of the reference standard. Thus, the terms “reference”, “Reference”, “reference subject” and “reference population” are used interchangeable herein. In one embodiment, the reference is a subject or a population of subjects having no said disease or condition, i.e., healthy controls or negative controls. In one embodiment, the reference is a subject or a population of subjects who develop said disease or condition at a later time. In one embodiment, the reference is a subject or a population of subjects having said disease or condition. In one embodiment, the reference is a subject or a population of subjects who have a disease described herein and have received a therapeutic or surgical treatment. In another embodiment, the reference is a subject or population of subjects with ADPKD. In yet another embodiment, the reference is a subject or population of subjects with CKD. In still another embodiment, the reference is a subject or a population of subjects having CKD which is not caused by ADPKD. In still another embodiment, the reference is a subject or a population of subjects who have high risks in developing CKD. In another embodiment, the reference is a subject or a population of subjects who have CKD caused by high blood pressure, type 1 diabetes, type 2 diabetes, glomerulonephritis, Interstitial nephritis, Polycystic kidney disease, Prolonged obstruction of the urinary tract, from conditions such as enlarged prostate, kidney stones and some cancers, Vesicoureteral reflux or Recurrent kidney infection (pyelonephritis). In still another embodiment, the reference is a subject or a population of subjects who have high blood pressure, type 1 diabetes, type 2 diabetes, glomerulonephritis, Interstitial nephritis, Polycystic kidney disease, Prolonged obstruction of the urinary tract, from conditions such as enlarged prostate, kidney stones and some cancers, Vesicoureteral reflux or Recurrent kidney infection (pyelonephritis). In one embodiment, the reference is a subject which is the same as the one to be diagnosed, detected or monitored for a disease described herein while the sample thereof was harvested at a different time, such as before, during or after a therapeutic or surgical treatment; or before, during or after an adjust of a therapeutic or surgical treatment. In one embodiment, the reference is a subject or a population of subjects who have preeclampsia. In another embodiment, the reference is a subject or a population of subjects who do not have preeclampsia. In yet another embodiment, the reference is a subject or a population of subjects who had preeclampsia. In yet another embodiment, the reference is a subject or a population of subjects who develop preeclampsia at a later time. In another embodiment, the reference is a subject or a population of subjects who was at an earlier time in the pregnancy and did not develop preeclampsia during the pregnancy. In yet another embodiment, the reference is a subject or a population of subjects who was at an earlier time in the pregnancy and developed preeclampsia later in the pregnancy.

Selection of the particular class of reference standards, reference population, Target or profile of Targets depends upon the use to which the diagnostic/monitoring methods and compositions are to be put by the physician and the desired result, e.g., diagnosis of a disease described herein at early stage, clinical management of patients with a disease described herein after a therapeutic treatment or an adjust thereof, including, but not limited to, monitoring for reoccurrence of disease or monitoring remission or progression of the disease and either before, during or after therapeutic or surgical intervention, selecting among therapeutic protocols for individual patients, monitoring for development of toxicity or other complications of therapy, predicting development of therapeutic resistance, and the like. Such reference standards or controls are the types that are commonly used in other similar methods.

A change in protein level of a Target required for diagnosis or detection by the methods described herein refers to a Target whose absolute protein level is increased or decreased in a subject or whose relative level over another molecule (such as another Target, or creatinine) is increased or decreased in a subject compared to that of a reference standard. The protein levels of a Target differ between normal subjects and subjects suffering from a disease (such as CKD, ADPKD, preeclampsia), between subjects who will not develop a disease as described herein and subjects who will develop the disease, between various stages of the same disease, or during progression of the same disease. Protein levels of a Target differ between pre-treatment and post-treatment patients with a disease described herein. Protein levels of a Target differ among patients treated with various treatments. Protein levels of a Target differ among patients with CKD of different causes. Such differences in protein levels of a Target or a combination of Targets include both quantitative, as well as qualitative, differences in the temporal or relative protein level, for example, samples of normal and diseased subjects, or among samples which have undergone different disease stages or different treatments.

For the purpose of the methods described herein, a significant change in Target levels when compared to that of a reference standard is considered to be present when there is a statistically significant difference in the Target level between the subject and reference, or when there is a statistical significant difference relative to a predetermined cut-point. Statistical significance may be determined by t-test, analysis of variance (ANOVA), multivariate techniques (e.g. MANOVA, ASCA, PCA, PLS) or other statistical methods known in the art. For example, in one embodiment, an increase of about 100 folds, about 75 folds, about 50 folds, about 30 folds, about 20 folds, about 15 folds, about 10 folds, about 9 folds, about 8 folds, about 7 folds, about 6 folds, about 5 folds, about 4 folds, about 3 folds, about 2 folds, about 100%, about 90%, about 80%, about 70%, about 60%, about 50%, about 40%, about 30%, about 20%, about 10%, about 5% relevant to the Target level of the reference is utilized as a predetermined cut-point. In another embodiment, a decrease of about 99%, about 95%, about 90%, about 80%, about 70%, about 60%, about 50%, about 40%, about 30%, about 20%, about 10%, about 5% relevant to the Target level of the reference is utilized as a predetermined cut-point.

As used herein, the “conservative amino acid replacement” or “conservative amino acid substitutions” refers to a change, replacement or substitution of an amino acid to a different amino acid with similar biochemical properties (e.g. charge, hydrophobicity and size), which is known by practitioners of the art. Also see, e.g. FRENCH et al. 1983, and YAMPOLSKY et al. 2005.

As used herein, a Coating Peptide refers to any naturally occurring or synthetic or recombinant peptide designed to be immobilized on an immobilization substrate or to be associated with a detectable label. Such Coating Peptide is capable of specifically complexing with or binding to a Ligand of a Target, while said Coating Peptide and Target may not simultaneously complex with or bind to a Ligand of the Target.

It should be understood that while various embodiments in the specification are presented using “comprising” language, under various circumstances, a related embodiment is also described using “consisting of” or “consisting essentially of” language. “Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

It is to be noted that the term “a” or “an”, refers to one or more, for example, “a Target”, is understood to represent one or more Target(s). As such, the terms “a” (or “an”), “one or more,” and “at least one” is used interchangeably herein.

As used herein, the term “about” means a variability of plus or minus 10% from the reference given, unless otherwise specified.

The terms “first” and “second” or “additional” are used throughout this specification as reference terms to distinguish between various forms and components of the compositions and methods.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 15 amino acids, about 150 amino acids, or a peptide fragment thereof or the corresponding nucleic acid sequence coding sequencers. A suitable amino acid fragment may be at least about 4 amino acids in length, and may be up to about 200 or up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., (THOMPSON et al. 1999).

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### II. Target

As used herein, the term “Target” refers to any naturally occurring or synthetic or recombinant amino acid sequence that is capable of specifically complexing with or binding to the Ligand as described herein. Specific embodiments of the Target are defined in detail below.

RhoB (Ras Homolog Family Member B) is a protein which in humans is encoded by the RHOB gene. Diseases associated with RhoB include Sertoli Cell-Only Syndrome, autosomal-dominant polycystic kidney disease, chronic kidney diseases, kidney dysfunction, or preeclampsia. Among its related pathways are Signaling by GPCR and Fc-GammaR Pathway. GO annotations related to this gene include GTP binding and GDP binding. RhoB has been found to mediate apoptosis in neoplastically transformed cells after DNA damage. RhoB is not essential for development but affects cell adhesion and growth factor signaling in transformed cells. Researchers have also shown that RhoB plays a negative role in tumorigenesis as deletion causes tumor formation, is involved in intracellular protein trafficking of a number of proteins, targets PKN1 to endosomes, is involved in trafficking of the EGF receptor from late endosomes to lysosomes, is required for stability and nuclear trafficking of AKT1/AKT which promotes endothelial cell survival during vascular development, serves as a microtubule-dependent signal that is required for the myosin contractile ring formation during cell cycle cytokinesis, and is required for genotoxic stress-induced cell death in breast cancer cells. The amino acid sequence for RhoB, which is reproduced herein as SEQ ID NO: 1, is publicly available, see, e.g., UniProtKB P62745 as well as NCBI Reference Sequence: NP 004031.1. In another embodiment, one or more of the RhoB fragments are also useful as Target(s) in the compositions and methods described herein, optionally in combination with one or more of the Targets described herein. It should be understood that, depending upon the context, any reference to RhoB herein also refers to any of these peptides.

In one embodiment, the Target of the compositions and methods described herein is a RhoB protein fragment having an amino acid sequence of SEQ ID NO: 1, a protein of at least 90% sequence identity thereof, or a protein having one or more conservative amino acid replacements thereof. In one embodiment, the Target is the amino acid sequence of SEQ ID NO: 2, a protein of at least 90% sequence identity thereof, or a protein having one or more conservative amino acid replacements thereof. In another embodiment, the Target is the amino acid sequence of SEQ ID NO: 3, a protein of at least 90% sequence identity thereof, or a protein having one or more conservative amino acid replacements thereof. In a further embodiment, the Target is a peptide fragment of SEQ ID NO: 1 with a length of at least 4 amino acids, a peptide of at least 90% sequence identity thereof, or a peptide having one or more conservative amino acid replacements thereof. In a further embodiment, the Target is a peptide fragment of SEQ ID NO: 1 with a length of about 4 amino acids to about 10, about 15, about 20, about 30, about 40, about 50, about 75, about 100, about 150, about 175 consecutive amino acids, a peptide of at least 90% sequence identity thereof, or a peptide having one or more conservative amino acid replacements thereof. In a further embodiment, the Target is a peptide fragment of SEQ ID NO: 1 with a length of up to about 190 consecutive amino acids, a peptide of at least 90% sequence identity thereof, or a peptide having one or more conservative amino acid replacements thereof. In one embodiment, the Target is RTDDGRAMAVRIQAYDYLE, SEQ ID NO: 4, a peptide of at least 90% sequence identity thereof, or a peptide having one or more conservative amino acid replacements thereof.

In one embodiment, the Target of the compositions and methods described herein is Xaa1Xaa2Xaa3IQAYDYLEXaa4Xaa5Xaa6Xaa7, or Xaa1Xaa2Xaa3IEAYDYLEXaa4Xaa5Xaa6Xaa7, or a peptide of at least 90% sequence identity thereof, or a peptide having one or more conservative amino acid replacements thereof, wherein (a) Xaa1 is Alanine (Ala or A), or absent; (b) Xaa2 is Valine (Val or V), or Xaa2 is absent if Xaa1 is absent; (c) Xaa3 is Arginine (Arg or R), or Xaa3 is absent if Xaa1 and Xaa2 are absent; (d) Xaa7 is Lysine (Lys or K), or absent; (e) Xaa6 is Alanine (Ala or A), or Xaa6 is absent if Xaa7 is absent; (g) Xaa5 is Serine (Ser or S), or Xaa5 is absent if Xaa6 and Xaa7 are absent; and (0 Xaa4 is Cysteine (Cys or C), or Xaa4 is absent if Xaa5, Xaa6 and Xaa7 are absent. In one embodiment, the Target is AVRIQAYDYLE, SEQ ID NO: 4, a peptide of at least 90% sequence identity thereof, or a peptide having one or more conservative amino acid replacements thereof. In another embodiment, the Target is IQAYDYLECSAK, SEQ ID NO: 8, a peptide of at least 90% sequence identity thereof, or a peptide having one or more conservative amino acid replacements thereof. In yet another embodiment, the Target is IEAYDYLECSAK, SEQ ID NO: 9, a peptide of at least 90% sequence identity thereof, or a peptide having one or more conservative amino acid replacements thereof. In a further embodiment, the Target is a peptide having an amino acid sequence listed in Table 1, a peptide of at least 90% sequence identity thereof, or a peptide having one or more conservative amino acid replacements thereof.

TABLE 1

| Peptide | SEQ ID NO |
|---|---|
| IQAYDYLE | 10 |
| RIQAYDYLE | 11 |
| VRIQAYDYLE | 12 |
| AVRIQAYDYLE | 7 |
| IQAYDYLEC | 13 |
| RIQAYDYLEC | 14 |
| VRIQAYDYLEC | 15 |
| AVRIQAYDYLEC | 16 |
| IQAYDYLECS | 17 |
| RIQAYDYLECS | 18 |
| VRIQAYDYLECS | 19 |
| AVRIQAYDYLECS | 20 |
| IQAYDYLECSA | 21 |
| RIQAYDYLECSA | 22 |
| VRIQAYDYLECSA | 23 |
| AVRIQAYDYLECSA | 24 |
| IQAYDYLECSAK | 8 |
| RIQAYDYLECSAK | 25 |
| VRIQAYDYLECSAK | 26 |
| AVRIQAYDYLECSAK | 27 |
| IEAYDYLE | 28 |
| RIEAYDYLE | 29 |
| VRIEAYDYLE | 30 |
| AVRIEAYDYLE | 31 |
| IEAYDYLEC | 32 |
| RIEAYDYLEC | 33 |
| VRIEAYDYLEC | 34 |
| AVRIEAYDYLEC | 35 |
| IEAYDYLECS | 36 |
| RIEAYDYLECS | 37 |
| VRIEAYDYLECS | 38 |
| AVRIEAYDYLECS | 39 |
| IEAYDYLECSA | 40 |
| RIEAYDYLECSA | 41 |
| VRIEAYDYLECSA | 42 |
| AVRIEAYDYLECSA | 43 |
| IEAYDYLECSAK | 9 |
| RIEAYDYLECSAK | 44 |
| VRIEAYDYLECSAK | 45 |
| AVRIEAYDYLECSAK | 46 |

In still other embodiments, the Target for use in the methods and compositions described herein can include various combinations of these Targets and/or fragments thereof.

### III. Diagnostic Reagents

In one aspect, a diagnostic reagent for use in the compositions and methods described herein is provided herein comprising a Ligand which is covalently linked to a detectable label or immobilized on an immobilization substrate and which is capable of to specifically complexing with, binding to, identifying or quantitatively detecting a Target within a RhoB protein. In one embodiment, the Ligand is an antibody or a fragment thereof. In another embodiment, the Ligand is an antibody comprising SEQ ID NOs: 48 and 50, or a modified molecule thereof. In yet another embodiment, the Ligand is an antibody comprising SEQ ID NOs: 52 and 54, or a modified molecule thereof.

In one embodiment, the antibody and antibody fragment of the instant invention may comprise at least one domain from the anti-RhoB monoclonal antibodies 7F7 and 9G5. In one embodiment, the Ligand comprises antibody 7F7. In another embodiment, the Ligand comprises antibody 9G5. Sequence of the light chain of antibody 7F7 is reproduced as SEQ ID NO: 48. In a further embodiment, the nucleic acid sequence encoding the light chain of antibody 7F7 is reproduced as SEQ ID NO: 47. Sequence of the heavy chain of antibody 7F7 is reproduced as SEQ ID NO: 50. In a further embodiment, the nucleic acid sequence encoding the heavy chain of antibody 7F7 is reproduced as SEQ ID NO: 49. Sequence of the light chain of antibody 9G5 is reproduced as SEQ ID NO: 52. In a further embodiment, the nucleic acid sequence encoding the light chain of antibody 9G5 is reproduced as SEQ ID NO: 51. Sequence of the heavy chain of antibody 9G5 is reproduced as SEQ ID NO: 54. In a further embodiment, the nucleic acid sequence encoding the heavy chain of antibody 9G5 is reproduced as SEQ ID NO: 54. For example, the antibody or antibody fragment may comprise at least one, two, three, four, five, or all six complementarity-determining region (CDR) domains of the anti-RhoB monoclonal antibodies 7F7 and 9G5. See Table 2 below for the CDRs of antibody 7F7 and antibody 9G5. In a particular embodiment, the antibody or antibody fragment comprises at least one or both of the CDR3 domains. In a particular embodiment, the domains of the antibody or antibody fragment have at least 90%, 95%, 97%, 99%, or 100% homology or identity with the domains present in the anti-RhoB monoclonal antibody 7F7 or 9G5. The domains may be longer or shorter than the domains depicted in Table 2 and SEQ ID No: 48, 50, 52, and 54 by about 1, 2, 3, 4, or 5, amino acids, particularly 1 or 2 amino acids, at the N-terminus and/or C-terminus of the domain.

TABLE 2

| CDR | Amino Acid Sequence | Sequence Listing |
|---|---|---|
| Light chain of 7F7 | RSSQSLVHSNGNTYLH | amino acid 26 to amino acid |
| CDR1 | | 41 of SEQ ID NO: 48 |
| Light chain of 7F7 | KVSNRFS | amino acid 57 to amino acid |
| CDR2 | | 63 of SEQ ID NO: 48 |
| Light chain of 7F7 | SQSTHVPYTFGGGTKLEIK | amino acid 96 to amino acid |
| CDR3 | | 114 of SEQ ID NO: 48 |
| Heavy chain of 7F7 | SYYMF | amino acid 24 to amino acid |
| CDR1 | | 28 of SEQ ID NO: 50 |
| Heavy chain of 7F7 | GFNPTNGGTDFNEKFKS | amino acid 43 to amino acid |
| CDR2 | | 59 of SEQ ID NO: 50 |
| Heavy chain of 7F7 | DGNLWGQGTSVTVSS | amino acid 101 to amino acid |
| CDR3 | | 115 of SEQ ID NO: 50 |
| Light chain of 9G5 | SASSSVSYMH | amino acid 26 to amino acid |
| CDR1 | | 35 of SEQ ID NO: 52 |
| Light chain of 9G5 | DTSNLAS | amino acid 51 to amino acid |
| CDR2 | | 57 of SEQ ID NO: 52 |
| Light chain of 9G5 | HQRSSYPYTFGGGTKLEIKR | amino acid 90 to amino acid |
| CDR3 | | 109 of SEQ ID NO: 52 |
| Heavy chain of 9G5 | TYAMN | amino acid 31 to amino acid |
| CDR1 | | 35 of SEQ ID NO: 54 |
| Heavy chain of 9G5 | RIRSKSNNYATYYADSVKD | amino acid 50 to amino acid |
| CDR2 | | 68 of SEQ ID NO: 54 |
| Heavy chain of 9G5 | GGGNLDYWGQGTTLTVSS | amino acid 101 to amino acid |
| CDR3 | | 118 of SEQ ID NO: 54 |

In one embodiment, the Ligand is capable of specifically complexing with, binding to, identifying or quantitatively detecting a single Target within a RhoB protein. In another embodiment, the Ligand is multi-specific to two or more Targets. In yet another embodiment, the Ligand for use in the methods and compositions described herein can include various combinations of these Ligands. In a further embodiment, each of these Ligands is capable of specifically complexing with, binding to, identifying or quantitatively detecting a single Target within a RhoB protein.

In a further embodiment, the Ligand is in a solution comprising stabilizer, diluent or other pharmaceutical acceptable carrier.

In still another embodiment, combinations of such labeled or immobilized Ligands are suitable reagents and components of a diagnostic kit.

### IV. Kits

In one aspect, provided herein is a diagnostic kit comprising (a) a First Ligand which is capable of specifically complexing with, binding to, identifying or quantitatively detecting a Target within a RhoB protein; and (b) a Coating Peptide which is RhoB protein or a fragment thereof, associated with a detectable label or immobilized on an immobilization substrate and capable of complexing with, binding to the First Ligand. In a further embodiment, the First Ligand is covalently linked to a detectable label or immobilized on an immobilization substrate. In another embodiment, a Second Ligand which is associated with a detectable label or is immobilized on an immobilization substrate and which is capable of specifically complexing with, binding to, identifying or quantitatively detecting the First Ligand. In yet another embodiment, the First Ligand or Second Ligand is a Ligand described herein.

In one embodiment, the Coating Peptide is a peptide fragment of SEQ ID NO: 1 with a length of about 6 amino acids to about 30 amino acids, a peptide of at least 90% sequence identity thereof, or a peptide having one or more conservative amino acid replacements thereof. In a further embodiment, the Coating Peptide is RTDDGRAMAVRIQAYDYLE, SEQ ID NO: 4, a peptide of at least 90% sequence identity thereof, or a peptide having one or more conservative amino acid replacements thereof. In yet another embodiment, the kit further comprises standard solution, blocking buffer, diluent, enzyme substrate, color development solution, stopping solution, mounting reagent and washing buffer.

In another aspect, provided herein is a diagnostic kit comprising a Ligand immobilized on an immobilization substrate and capable of specifically complexing with, binding to, identifying or quantitatively detecting a Target within a RhoB protein. In one embodiment, said Ligand is as described herein. In one embodiment, the immobilization substrate is a chromatography column, a bead, a magnetic particle, a multi-well plate, a microarray, a microfluidics card, a chip, or a chamber. In a further embodiment, the kit further comprises standard solution, blocking buffer, diluent, stabilizer, washing buffer, elution buffer, centrifugal filter concentrators, membranes for dialysis and dialysis buffer. Any combination of the above-described labeled or immobilized Ligands can be assembled in a diagnostic kit described herein. For example, one embodiment of a diagnostic kit includes a Ligand which is capable of specifically complexing with, binding to, identifying or quantitatively detecting a first Target within a RhoB protein.

Another embodiment of a diagnostic kit includes a Ligand which is capable of specifically complexing with, binding to, identifying or quantitatively detecting a second Target within a RhoB protein. Still a Ligand which is capable of specifically complexing with, binding to, identifying or quantitatively detecting an additional Target within a RhoB provide additional diagnostic kits.

For these reagents, the labels may be selected from among many known diagnostic labels, including those described above. Similarly, the immobilization substrates for immobilization may be any of the common immobilization substrates, including those described herein. The selection of suitable detectable labels and to immobilization substrates used in the reagents, kits and methods are routine determinations made by one of skill in the art in view of the teachings herein. Thus, a kit or device can contain multiple reagents or one or more individual reagents. For example, one embodiment of a composition includes an immobilization substrate upon which a Ligand is immobilized. In another embodiment, the kit also contains optional detectable labels, immobilization substrates, optional enzyme substrates for enzymatic labels, standard solution, blocking buffer, diluent, enzyme substrate, stopping solution, mounting reagent, washing buffer, stabilizer, washing buffer, elution buffer, centrifugal filter concentrators, membranes for dialysis, dialysis buffer, as well as other laboratory items. In one embodiment, the reagents and kits are designed for use in urine.

The diagnostic reagents or kits can be presented for use as assays formats such as a direct or indirect competitive ELISA, a direct or indirect competitive ELISPOT, a direct or indirect competitive MSD, a direct or indirect competitive fluorescent immunoassay, a direct or indirect competitive lateral flow test, a direct or indirect competitive dipstick test, a direct or indirect competitive lap-on-a-chip test, liquid chromatographic mass spectrometry, multiple reaction monitoring (MRM) mass spectrometry, or triple-quadrupole mass spectrometers, or platform multiplex ELISAs, such as the BioRad Luminex platform.

In another embodiment, the diagnostic kit is a lateral flow device comprising (a) a First Ligand which is capable of specifically complexing with, binding to, identifying or quantitatively detecting a Target within a RhoB protein; and (b) a Coating Peptide which is RhoB protein or a fragment thereof, associated with a detectable label and capable of complexing with, binding to the First Ligand. Formats and designs of lateral flow device is known to one of skills in the art. See, e.g. SAJID, et al. 2015. In a further embodiment, the First Ligand is immobilized on an immobilization substrate. In another embodiment, the diagnostic kit further comprises a Second Ligand which is immobilized on an immobilization substrate and which is capable of specifically complexing with, binding to, identifying or quantitatively detecting the First Ligand.

### V. Methods

In one aspect, provided herein is a method for diagnosing, detecting or monitoring the progress of a disease in a subject comprising contacting a sample obtained from a subject with a composition comprising: (i) a First Ligand which is capable of specifically to complexing with, binding to, identifying or quantitatively detecting a Target within a RhoB protein; and (ii) a Coating Peptide which is RhoB protein or a fragment thereof, immobilized on an immobilization substrate and capable of complexing with, binding to the First Ligand. In another step, the method involves detecting the presence or measuring the levels of one or more Targets; and comparing the Target levels in the subject's sample with the level in a reference standard. The presence, absence, appearance, disappearance, significant increase or significant decrease in Target level in the subject's sample compared to that in the reference standard indicates a diagnosis, risk, progression or remission of the disease in the subject.

In one embodiment, the method further comprises an additional step of contacting the sample with a Second Ligand which is associated with a detectable label and which is capable of specifically complexing with, binding to, identifying or quantitatively detecting the First Ligand. In another embodiment, the method comprises performing a direct or indirect competitive ELISA, a direct or indirect competitive ELISPOT, a direct or indirect competitive MSD, a direct or indirect competitive lateral flow test, a direct or indirect competitive dipstick test, a direct or indirect competitive lab-on-a-chip test, or a direct or indirect competitive fluorescent immunoassay.

In another aspect, provided herein is a method for diagnosing, detecting or monitoring the progress of a disease in a subject comprising performing co-immunoprecipitation (Co-IP), affinity chromatography and other pull-down assays via contacting a sample obtained from a subject with a composition comprising a Ligand capable of specifically complexing with, binding to, identifying or quantitatively detecting a Target within a RhoB protein to deplete non-Target molecule in the sample or to enrich the Targets. Another step in this method involves performing mass spectrometry, gel electrophoresis, or other protein quantitation assays to identify the pulled-down Targets identified above or to measure the Target levels, or ratios thereof. In certain embodiments, the method further involves a step of comparing the Target levels in the subject's sample with the level in a reference standard. The presence, absence, appearance, disappearance, significant increase or significant decrease in Target level in the subject's sample compared to that in the reference standard indicates a diagnosis, risk, progression or remission of the disease in the subject. In one embodiment, the method employs the technique of mass spectrometry, such as liquid chromatographic mass spectrometry. In another embodiment, the method involves multiple reaction monitoring to (MRM) mass spectrometry, or triple-quadrupole mass spectrometer. In another embodiment, the method further comprises fragmenting the pulled-down Target(s) with a chemical or enzymatic agent between the first contacting step and the identifying step. Conventional pull-down methods are well-known in the art. See, e.g. MEYSMAN, et al. 2015.

In one embodiment, the method described herein is for diagnosing or detecting a disease. In another embodiment, the method is designed for early diagnosis of disease. In another embodiment, the methods are used for determining the best clinical treatment. In still another embodiment, the methods are useful for monitoring relapse after initial diagnosis and treatment, or predicting clinical outcome.

A. Sample Preparation

The sample is obtained from a subject. The subject's sample can in one embodiment be provided before initial diagnosis or concurrently with other diagnostic method available clinically, so that the method is performed to diagnose a disease described herein. In another embodiment, depending upon the reference standard used, the method is performed to diagnosis the stage of a disease. In another embodiment, depending upon the reference standard used, the method is performed to diagnosis the cause of a disease. In another embodiment, the subject's sample can be provided after a diagnosis, so that the method is performed to monitor progression of a disease. In another embodiment, the sample can be provided prior to a therapeutic treatment or a surgical treatment and the method used to thereafter monitor the effect of the treatment or surgery, and to check for relapse. In a further embodiment, the surgical treatment is a kidney transplant. In another embodiment, the sample can be provided following a therapeutic treatment or a surgical treatment and the method performed to ascertain efficacy of treatment or relapse. In yet another embodiment the sample may be obtained from the subject periodically during a therapeutic treatment, and the method employed to track efficacy of therapy or relapse. In yet another embodiment the sample may be obtained from the subject periodically during therapeutic treatment to enable the physician to change or adjust therapies. In one or more of these embodiments, the subject's own prior sample can be employed in the method as the reference standard.

Preferably where the sample is a fluid, e.g., urine, blood, serum or plasma, obtaining the sample involves simply withdrawing and preparing the sample in traditional fashion for contact with the diagnostic reagent.

The method further involves contacting the sample obtained from a subject with a diagnostic reagent as described above under conditions that permit the reagent to bind to or complex with one or more Targets which may be present in the sample. This method may employ any of the suitable diagnostic reagents or kits or compositions described above.

In one embodiment, the method further includes enriching the Target(s) via conventional methods known in the art or via commercially available kits. For example, protein precipitation with a neutral salt, such as ammonium sulfate.

In another embodiment, the method further includes fragmenting the Target(s) by specific proteolysis with a chemical or enzymatic agent via conventional methods known in the art. In one embodiment, the method involves enriching the Target(s) or one or more peptides produced by specific proteolysis in the sample. In another embodiment, the method involves depleting the sample of non-target proteins. The depletion may also be performed using antibodies to the non-targets.

B. Measuring Biomarker Levels

After preparation of and contacting the sample as described above, a suitable assay is employed to detect or measure in the sample the protein level (actual or relative) of one or more Targets. Alternatively, a suitable assay is employed to generate a protein abundance profile (actual or relative or ratios thereof) of Targets from the sample. In another embodiment, the above method is combined with a diagnostic method known in the art.

The measurement of the Target(s) in the sample may employ any suitable Ligand described herein. In one embodiment, the Ligand is an antibody or a fragment thereof. Antibodies or fragments useful in the method may be generated synthetically or recombinantly, using conventional techniques or may be isolated and purified from plasma or further manipulated to increase the binding affinity thereof. It should be understood that any antibody, antibody fragment, or mixture thereof that binds one of the Target(s) may be employed in the methods described herein, regardless of how the antibody or mixture of antibodies was generated.

Similarly the particular assay format used to measure the Target(s) in a sample via a Ligand as described herein may be selected from among a wide range of protein assays, such as described in the examples below. Suitable assays may also include a direct or indirect competitive ELISA, a direct or indirect competitive ELISPOT, a direct or indirect competitive MSD or a direct or indirect fluorescent immunoassay, enzyme-linked immunoassays, sandwich immunoassays, homogeneous assays, immunohistochemistry formats, lateral flow assays, dipstick assays, lab-on-a-chip assays, or other conventional assay formats. In another embodiment, a mass spectrometry-based assay is employed. In another embodiment, a liquid chromatographic mass spectrometry, multiple reaction monitoring (MRM) mass spectrometry, or triple-quadrupole mass spectrometer is employed, in which Ligand(s) are used to enrich the Target(s) in a manner analogous to the capture antibody in sandwich ELISAs. One of skill in the art may readily select from any number of conventional immunoassay formats to perform this invention.

Other reagents for the detection of protein in samples, such as synthetic chemical compounds capable of detecting the Target(s) may be used in other assay formats for the quantitative detection of protein in biological samples, such as high pressure liquid chromatography (HPLC), Fast protein liquid chromatography (FPLC) etc.

C. Detection of a Change in Biomarker Abundance Level and Diagnosis

The protein level of the one or more Target(s) in the subject's sample or the protein abundance profile of multiple said Targets that is detected by the use of the methods and compositions described above is then compared with the level of the same Target or Targets in a reference standard or reference profile. In one embodiment, the comparing step of the method is performed by a computer processor or computer-programmed instrument that generates numerical or graphical data useful in the appropriate diagnosis of the disease. Optionally, the comparison may be performed manually.

The detection or observation of a change in the protein level of a Target or Targets in the subject's sample from the same Target or Targets in the reference standard can indicate an appropriate diagnosis. An appropriate diagnosis can be identifying a risk of developing a disease, a diagnosis of a disease (or stage thereof), a diagnosis of the cause of a disease, a diagnosis or detection of the status of progression or remission of a disease in the subject following therapy or surgery, a determination of the need for a change in therapy or an adjust in dosage of therapeutic agent. The method is thus useful for early diagnosis of disease, for monitoring response or relapse after initial diagnosis and treatment or to predict clinical outcome or determine the best clinical treatment for the subject.

In one embodiment, the change in protein level of a Target in a subject is an increase in comparison to that in the reference standard. In another embodiment, the change in protein level of a Target is a decrease in comparison to that in the reference standard.

The results of the methods and use of the compositions described herein may be used in conjunction with clinical risk factors or result of other available clinical tests to help physicians make more accurate decisions about how to manage patients with a disease described herein. Another advantage of these methods and compositions is that diagnosis may occur earlier than those with more invasive diagnostic measures.

### VI. Examples

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1—the RhoB Antibody 7F7 Identifies Fragments of RhoB Protein

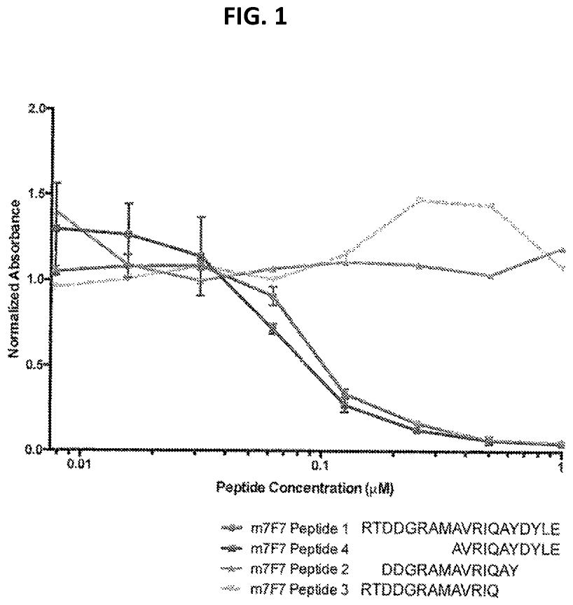

To identify the amino acids recognized by the RhoB antibody 7F7 as well as to assess the binding between the RhoB antibody 7F7 and various fragments of RhoB protein, competitive ELISA as described herein was performed. Briefly, each well on an ELISA plate was coated with peptide with an amino acid sequence of Acetyl-RTDDGRAMAVRIQAYDYLE-Amidyl (SEQ ID NO: 4). After blocking, four competing peptides (RhoB Peptide 1: RTDDGRAMAVRIQAYDYLE, SEQ ID NO: 4; RhoB Peptide 4: AVRIQAYDYLE, SEQ ID NO: 7; RhoB Peptide 2: DDGRAMAVRIQAY, SEQ ID NO: 5; and RhoB Peptide 3: RTDDGRAMAVRIQ, SEQ ID NO: 6) were incubated at various concentrations in the presence of the tested RhoB antibody 7F7. Horseradish peroxidase enzyme (HRP) conjugated secondary antibody recognizing 7F7 was applied to each well after appropriate washes. Chromogenic enzyme substrates were provided to producing a quantitative signal measured by a plate reader. A decline in absorbance was observed upon increasing concentrations of RhoB Peptides 1 and 4 but not 2 or 3, indicating epitope of antibody 7F7 located in RhoB Peptides 1 and 4 but not in RhoB Peptides 2 or 3 (FIG. 1).

### Example 2—The RhoB Antibody 7F7 Identifies CKD-related Fragments of RhoB Protein

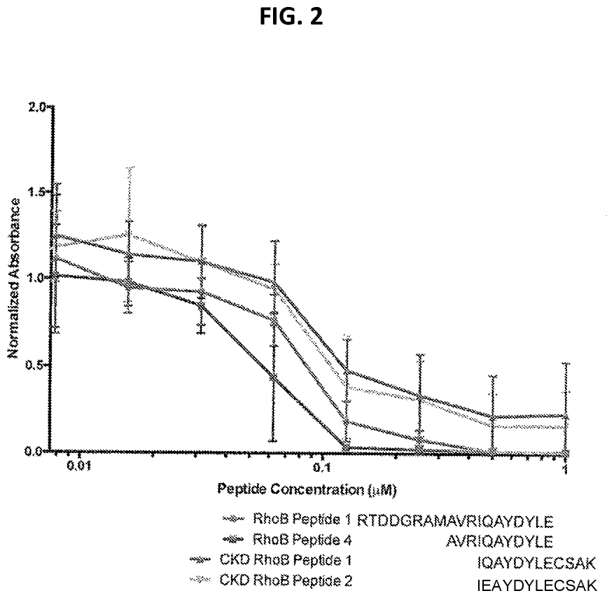

Competitive ELISA as described in Example 1 was performed. CDK RhoB Peptides 1 (SEQ ID NO: 8) and 2 (SEQ ID NO: 9) were utilized as competing peptides. RhoB Peptides 1 and 4 were provided as positive controls. The data acquired is shown in FIG. 2. The result demonstrated that the RhoB antibody 7F7 successfully identified CDK RhoB Peptides 1 and 2, indicating an application as a diagnostic reagent for CDK.

### Table 3. Sequence Listing Free Text

The following information is provided for sequences containing free text under numeric identifier <223>.

| SEQ ID NO: | |
|---|---|
| (containing | |
| free text) | Free Text under <223> |
| 28 | <213> Artificial Sequence |
| | <220> |
| | <223> Synthetic amino acid sequence |
| 29 | <213> Artificial Sequence |
| | <220> |
| | <223> Synthetic amino acid sequence |
| 30 | <213> Artificial Sequence |
| | <220> |
| | <223> Synthetic amino acid sequence |
| 31 | <213> Artificial Sequence |
| | <220> |
| | <223> Synthetic amino acid sequence |
| 32 | <213> Artificial Sequence |
| | <220> |
| | <223> Synthetic amino acid sequence |
| 33 | <213> Artificial Sequence |
| | <220> |
| | <223> Synthetic amino acid sequence |
| 34 | <213> Artificial Sequence |
| | <220> |
| | <223> Synthetic amino acid sequence |
| 35 | <213> Artificial Sequence |
| | <220> |
| | <223> Synthetic amino acid sequence |
| 36 | <213> Artificial Sequence |
| | <220> |
| | <223> Synthetic amino acid sequence |
| 37 | <213> Artificial Sequence |
| | <220> |
| | <223> Synthetic amino acid sequence |
| 38 | <213> Artificial Sequence |
| | <220> |
| | <223> Synthetic amino acid sequence |
| 39 | <213> Artificial Sequence |
| | <220> |
| | <223> Synthetic amino acid sequence |
| 40 | <213> Artificial Sequence |
| | <220> |
| | <223> Synthetic amino acid sequence |
| 41 | <213> Artificial Sequence |
| | <220> |
| | <223> Synthetic amino acid sequence |
| 42 | <213> Artificial Sequence |
| | <220> |
| | <223> Synthetic amino acid sequence |
| 43 | <213> Artificial Sequence |
| | <220> |
| | <223> Synthetic amino acid sequence |
| 44 | <213> Artificial Sequence |
| | <220> |
| | <223> Synthetic amino acid sequence |
| 45 | <213> Artificial Sequence |
| | <220> |
| | <223> Synthetic amino acid sequence |
| 46 | <213> Artificial Sequence |
| | <220> |
| | <223> Synthetic amino acid sequence |
| 47 | 213> Artificial Sequence |
| | <220> |
| | <223> light chain of 7F7 |
| | <221> misc_feature |
| | <222> (76) . . . (121) |
| | <223> CDR1 |
| | <221> misc_feature |
| | <222> (169) . . . (187) |
| | <223> CDR2 |
| | <221> misc_feature |
| | <222> (286) . . . (340) |
| | <223> CDR2 |
| 48 | <213> Artificial Sequence |
| | <223> light chain of 7F7 |
| | <221> MISC_FEATURE |
| | <222> (26) . . . (41) |
| | <223> CDR1 |
| | <221> MISC_FEATURE |
| | <222> (57) . . . (63) |
| | <223> CDR2 |
| | <221> MISC_FEATURE |
| | <222> (96) . . . (114) |
| | <223> CDR2 |
| 49 | <213> Artificial Sequence |
| | <220> |
| | <223> heavy chain of 7F7 |
| | <220> |
| | <221> misc_feature |
| | <222> (70) . . . (82) |
| | <223> CDR1 |
| | <220> |
| | <221> misc_feature |
| | <222> (127) . . . (175) |
| | <223> CDR2 |
| | <220> |
| | <221> misc_feature |
| | <222> (301) . . . (343) |
| | <223> CDR3 |
| 50 | <213> Artificial Sequence |
| | <220> |
| | <223> heavy chain of 7F7 |
| | <220> |
| | <221> MISC_FEATURE |
| | <222> (24) . . . (28) |
| | <223> CDR1 |
| | <220> |
| | <221> MISC_FEATURE |
| | <222> (43) . . . (59) |
| | <223> CDR2 |
| | <220> |
| | <221> MISC_FEATURE |
| | <222> (100) . . . (115) |
| | <223> CDR2 |
| 51 | <213> Artificial Sequence |
| | <220> |
| | <223> light chain of 9G5 |
| | <220> |
| | <221> misc_feature |
| | <222> (76) . . . (103) |
| | <223> CDR1 |
| | <220> |
| | <221> misc_feature |
| | <222> (151) . . . (169) |
| | <223> CDR2 |
| | <220> |
| | <221> misc_feature |
| | <222> (268) . . . (325) |
| | <223> CDR3 |
| 52 | <213> Artificial Sequence |
| | <220> |
| | <223> light chain of 9G5 |
| | <220> |
| | <221> MISC_FEATURE |
| | <222> (26) . . . (35) |
| | <223> CDR1 |
| | <220> |
| | <221> MISC_FEATURE |
| | <222> (51) . . . (57) |
| | <223> CDR2 |
| | <220> |
| | <221> MISC_FEATURE |
| | <222> (90) . . . (109) |
| | <223> CDR3 |
| 53 | <213> Artificial Sequence |
| | <220> |
| | <223> heavy chain of 9G5 |
| | <220> |
| | <221> misc_feature |
| | <222> (91) . . . (103) |
| | <223> CDR1 |
| | <220> |
| | <221> misc_feature |
| | <222> (148) . . . (202) |
| | <223> CDR2 |
| | <220> |
| | <221> misc_feature |
| | <222> (301) . . . (352) |
| | <223> CDR3 |
| 54 | <213> Artificial Sequence |
| | <220> |
| | <223> heavy chain of 9G5 |
| | <220> |
| | <221> MISC_FEATURE |
| | <222> (31) . . . (35) |
| | <223> CDR1 |
| | <220> |
| | <221> MISC_FEATURE |
| | <222> (50) . . . (68) |
| | <223> CDR2 |
| | <220> |
| | <221> MISC_FEATURE |
| | <222> (101) . . . (118) |
| | <223> CDR3 |

Each and every patent, patent application, and publication, including publications listed herein, and publicly available peptide sequences cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

### VII. References

ABBOUD, et al. (2012). “Clinical Practice Guideline for the Evaluation and Management of Chronic Kidney Disease.” Journal of the International Society of Nephrology. KDIGO: 5-119.
CRAICI, et al. (2013). “Podocyturia predates proteinuria and clinical features of preeclampsia: longitudinal prospective study.” Hypertension 61(6): 1289-1296.
DESMET, et al. (2014). “Structural basis of IL-23 antagonism by an Alphabody protein scaffold.” Nature communications 5.
EBERSBACH, et al. (2007). “Affilin-novel binding molecules based on human γ-b-crystallin, an all (3-sheet protein.” Journal of molecular biology 372(1): 172-185.
FRENCH, et al. (1983). “What is a conservative substitution?” Journal of molecular Evolution 19(2): 171-175.
GRABULOVSKI, et al. (2007). “A novel, non-immunogenic Fyn SH3-derived binding protein with tumor vascular targeting properties.” Journal of Biological Chemistry 282(5): 3196-3204.
HAN, et al. (2016). “RhoB/ROCK mediates oxygen-glucose deprivation-stimulated syncytiotrophoblast microparticle shedding in preeclampsia.” Cell Tissue Res 366(2): 411-425.
JAYASENA (1999). “Aptamers: an emerging class of molecules that rival antibodies in diagnostics.” Clinical chemistry 45(9): 1628-1650.
JOHNSON, et al. (2012). “Sensitive affimer and antibody based impedimetric label-free assays for C-reactive protein.” Analytical chemistry 84(15): 6553-6560.
KAWE, et al. (2006). “Isolation of intracellular proteinase inhibitors derived from designed ankyrin repeat proteins by genetic screening.” Journal of Biological Chemistry 281(52): 40252-40263.
KIM, et al. (2013). “Protein immobilization techniques for microfluidic assays.” Biomicrofluidics 7(4): 041501.
KOIDE, et al. (2007). “Monobodies: antibody mimics based on the scaffold of the fibronectin type III domain.” Protein Engineering Protocols: 95-109.
KONVALINKA, et al. (2016). “Quantification of angiotensin II-regulated proteins in urine of patients with polycystic and other chronic kidney diseases by selected reaction monitoring.” Clin Proteomics 13: 16.
KONVALINKA, et al. (2013). “Determination of an angiotensin II-regulated proteome in primary human kidney cells by stable isotope labeling of amino acids in cell culture (SILAC).” J Biol Chem 288(34): 24834-24847.
LÖFBLOM, et al. (2010). “Affibody molecules: engineered proteins for therapeutic, diagnostic and biotechnological applications.” FEBS letters 584(12): 2670-2680.
MEYSMAN, et al. (2015). “Protein complex analysis: From raw protein lists to protein interaction networks.” Mass spectrometry reviews.
MOURATOU, et al. (2015). “Artificial affinity proteins as ligands of immunoglobulins.” Biomolecules 5(1): 60-75.
NIXON, et al. (2006). “Engineered protein inhibitors of proteases.” Current opinion in drug discovery & development 9(2): 261-268.
SAJID, et al. (2015). “Designs, formats and applications of lateral flow assay: A literature review.” Journal of Saudi Chemical Society 19(6): 689-705.
SILVERMAN, et al. (2005). “Multivalent avimer proteins evolved by exon shuffling of a family of human receptor domains.” Nature biotechnology 23(12): 1556-1561.
SKERRA (2008). “Alternative binding proteins: anticalins-harnessing the structural plasticity of the lipocalin ligand pocket to engineer novel binding activities.” FEBS journal 275(11): 2677-2683.
STUMPP, et al. (2008). “DARPins: a new generation of protein therapeutics.” Drug discovery today 13(15): 695-701.
TERSKIKH, et al. (1997). ““Peptabody”: a new type of high avidity binding protein.” Proceedings of the National Academy of Sciences 94(5): 1663-1668.
THOMPSON, et al. (1999). “A comprehensive comparison of multiple sequence alignment programs.” Nucleic acids research 27(13): 2682-2690.
YAMPOLSKY, et al. (2005). “The exchangeability of amino acids in proteins.” Genetics 170(4): 1459-1472.
ZYNDA, et al. (2016). “An RNA interference screen identifies new avenues for nephroprotection.” Cell Death Differ. 2016 April; 23(4):608-15. doi: 10.1038/cdd.2015.128. Epub 2015 Nov. 13.

1. A method for diagnosing, detecting or monitoring the progress of a disease in a subject comprising:
(a) contacting a sample obtained from a subject with a composition comprising:
(i) a First Ligand which is capable of specifically complexing with, binding to, identifying or quantitatively detecting a Target within a RhoB protein; and
(ii) a Coating Peptide which is RhoB protein or a fragment thereof,
immobilized on an immobilization substrate and capable of complexing with, binding to the First Ligand;
(b) detecting the presence or measuring the levels of one or more Targets; and
(c) comparing the Target levels in the subject's sample with the level in a reference standard;
wherein the presence, absence, a significant increase or a significant decrease in Target level in the subject's sample compared to that in the reference standard indicates a diagnosis, risk, progression or remission of the disease in the subject.

2. The method according to claim 1, further comprising at least one step selected from:
(d) after step (a), contacting the sample with a Second Ligand which is associated with a detectable label and which is capable of specifically complexing with, binding to, identifying or quantitatively detecting the First Ligand; and
(e) performing a direct or indirect competitive ELISA, a direct or indirect competitive ELISPOT, a direct or indirect competitive MSD or a direct or indirect fluorescent immunoassay.

3. (canceled)

4. The method according to claim 1, wherein the disease is autosomal-dominant polycystic kidney disease, other chronic kidney diseases, kidney dysfunction, or preeclampsia.

5-7. (canceled)

8. The method according to claim 1, wherein said Ligand is covalently linked to a detectable label or immobilized on an immobilization substrate and which is capable of specifically complexing with, binding to, identifying or quantitatively detecting a Target within a RhoB protein.

9-11. (canceled)

13. The method according to claim 1, wherein the First Ligand is an antibody comprising SEQ ID NOs: 48 and 50, or a modified molecule thereof; or wherein the First Ligand is an antibody comprising SEQ ID NOs: 52 and 54, or a modified molecule thereof.

14-18. (canceled)

19. The method according to claim 1, wherein
(a) said Target is a RhoB protein of fragment having an amino acid sequence of SEQ ID NO: 1, a protein of at least 90% sequence identity thereof, or a protein having one or more conservative amino acid replacements thereof; or
(b) said Target is the amino acid sequence of SEQ ID NO: 2, a protein of at least 90% sequence identity thereof, or a protein having one or more conservative amino acid replacements thereof; or
(c) said Target is the amino acid sequence of SEQ ID NO: 3, a protein of at least 90% sequence identity thereof, or a protein having one or more conservative amino acid replacements thereof; or
(d) said Target is a peptide fragment of SEQ ID NO: 1 with a length of at least 4 amino acids, a peptide of at least 90% sequence identity thereof, or a peptide having one or more conservative amino acid replacements thereof; or
(e) said Target is RTDDGRAMAVRIQAYDYLE, SEQ ID NO: 4, a peptide of at least 90% sequence identity thereof, or a peptide having one or more conservative amino acid replacements thereof; or
(f) said Target is AVRIQAYDYLE, SEQ ID NO: 7, a peptide of at least 90% sequence identity thereof, or a peptide having one or more conservative amino acid replacements thereof; or
(g) said Target is IQAYDYLECSAK, SEQ ID NO: 8, a peptide of at least 90% sequence identity thereof, or a peptide having one or more conservative amino acid replacements thereof; or
(h) said Target is IEAYDYLECSAK, SEQ ID NO: 9, a peptide of at least 90% sequence identity thereof, or a peptide having one or more conservative amino acid replacements thereof.

20-23. (canceled)

24. The method according to claim 1, wherein said target is Xaa1Xaa2Xaa3IQAYDYLEXaa4Xaa5Xaa6Xaa7, or Xaa1Xaa2Xaa3IEAYDYLEXaa4Xaa5Xaa6Xaa7, or a peptide of at least 90% sequence identity thereof, or a peptide having one or more conservative amino acid replacements thereof, wherein
(a) Xaa1 is Alanine (Ala or A), or absent;
(b) Xaa2 is Valine (Val or V), or Xaa2 is absent if Xaa1 is absent;
(c) Xaa3 is Arginine (Arg or R), or Xaa3 is absent if Xaa1 and Xaa2 are absent;
(d) Xaa7 is Lysine (Lys or K), or absent.
(e) Xaa6 is Alanine (Ala or A), or Xaa6 is absent if Xaa7 is absent;
(g) Xaa5 is Serine (Ser or S), or Xaa5 is absent if Xaa6 and Xaa7 are absent; and
(h) Xaa4 is Cysteine (Cys or C), or Xaa4 is absent if Xaa5, Xaa6 and Xaa7 are absent.

25-27. (canceled)

28. The method according to claim 1, wherein said Ligand is multi-specific to two or more Targets and each Target on a single Ligand is a different RhoB peptide.

29. A diagnostic reagent comprising a Ligand which is covalently linked to a detectable label or immobilized on an immobilization substrate and which is capable of specifically complexing with, binding to, identifying or quantitatively detecting a Target within a RhoB protein.

30-32. (canceled)

33. The reagent according to claim 29, wherein said Ligand is an antibody comprising SEQ ID NOs: 48 and 50, or a modified molecule thereof; or wherein said Ligand is an antibody comprising SEQ ID NOs: 52 and 54, or a modified molecule thereof.

34-38. (canceled)

39. The reagent according to claim 29, wherein said Target is:
(a) a RhoB protein of fragment having an amino acid sequence of SEQ ID NO: 1, a protein of at least 90% sequence identity thereof, or a protein having one or more conservative amino acid replacements thereof;
(b) the amino acid sequence of SEQ ID NO: 2, a protein of at least 90% sequence identity thereof, or a protein having one or more conservative amino acid replacements thereof;
(c) the amino acid sequence of SEQ ID NO: 3, a protein of at least 90% sequence identity thereof, or a protein having one or more conservative amino acid replacements thereof;
(d) a peptide fragment of SEQ ID NO: 1 with a length of at least 4 amino acids, a peptide of at least 90% sequence identity thereof, or a peptide having one or more conservative amino acid replacements thereof;
(e) RTDDGRAMAVRIQAYDYLE, SEQ ID NO: 4, a peptide of at least 90% sequence identity thereof, or a peptide having one or more conservative amino acid replacements thereof;
(f) Xaa1Xaa2Xaa3IQAYDYLEXaa4Xaa5Xaa6Xaa7, or
Xaa1Xaa2Xaa3IEAYDYLEXaa4Xaa5Xaa6Xaa7, or a peptide of at least 90% sequence identity thereof, or a peptide having one or more conservative amino acid replacements thereof, wherein
i. Xaa1 is Alanine (Ala or A), or absent;
ii. Xaa2 is Valine (Val or V), or Xaa2 is absent if Xaa1 is absent;
iii. Xaa3 is Arginine (Arg or R), or Xaa3 is absent if Xaa1 and Xaa2 are absent;
iv. Xaa7 is Lysine (Lys or K), or absent,
v. Xaa6 is Alanine (Ala or A), or Xaa6 is absent if Xaa7 is absent;
vi. Xaa5 is Serine (Ser or S), or Xaa5 is absent if Xaa6 and Xaa7 are absent; and
vii. Xaa4 is Cysteine (Cys or C), or Xaa4 is absent if Xaa5, Xaa6 and Xaa7 are absent;
(g) AVRIQAYDYLE, SEQ ID NO: 7, a peptide of at least 90% sequence identity thereof, or a peptide having one or more conservative amino acid replacements thereof;
(h) IQAYDYLECSAK, SEQ ID NO: 8, a peptide of at least 90% sequence identity thereof, or a peptide having one or more conservative amino acid replacements thereof; or
(i) IEAYDYLECSAK, SEQ ID NO: 9, a peptide of at least 90% sequence identity thereof, or a peptide having one or more conservative amino acid replacements thereof.

40-47. (canceled)

48. The reagent according to claim 29, wherein said Ligand is multi-specific to two or more Targets and each Target on a single Ligand is a different RhoB peptide.

49. A diagnostic kit comprising
(a) a First Ligand which is capable of specifically complexing with, binding to, identifying or quantitatively detecting a Target within a RhoB protein; and
(b) a Coating Peptide which is RhoB protein or a fragment thereof, associated with a detectable label or immobilized on an immobilization substrate and capable of complexing with, binding to the First Ligand.

50. The kit according to claim 49, wherein said First Ligand is covalently linked to a detectable label or immobilized on an immobilization substrate.

51. The kit according to claim 49, further comprising a Second Ligand which is associated with a detectable label and which is capable of specifically complexing with, binding to, identifying or quantitatively detecting the First Ligand.

52. (canceled)

53. The kit according to claim 49, wherein said Coating Peptide is:
(a) a peptide fragment of SEQ ID NO: 1 with a length of about 6 amino acids to about 30 amino acids, a peptide of at least 90% sequence identity thereof, or a peptide having one or more conservative amino acid replacements thereof; or
(b) RTDDGRAMAVRIQAYDYLE, SEQ ID NO: 4, a peptide of at least 90% sequence identity thereof, or a peptide having one or more conservative amino acid replacements thereof.

54-55. (canceled)

57. A diagnostic kit comprising a Ligand immobilized on an immobilization substrate and capable of specifically complexing with, binding to, identifying or quantitatively detecting a Target within a RhoB protein.

58-60. (canceled)

61. A method for diagnosing, detecting or monitoring the progress of a disease in a subject comprising:
(a) performing co-immunoprecipitation (Co-IP), affinity chromatography and other pull-down assays via contacting a sample obtained from a subject with a composition comprising a Ligand capable of specifically complexing with, binding to, identifying or quantitatively detecting a Target within a RhoB protein to deplete non-Target molecule in the sample or to enrich the Targets;
(b) performing mass spectrometry, gel electrophoresis, or other protein quantitation assays to identify the pulled-down Targets of (a) or to measure the Target levels, or ratios thereof;
(c) comparing the Target levels in the subject's sample with the level in a reference standard;
wherein the presence, absence, disappearance, significant increase or significant decrease in Target level in the subject's sample compared to that in the reference standard indicates a diagnosis, risk, progression or remission of the disease in the subject.

62. The method according to claim 61, comprising one or more of the features:
(d) wherein the mass spectrometry is liquid chromatographic mass spectrometry or multiple reaction monitoring (MRM) mass spectrometry;
(e) further comprising fragmenting the pulled-down Target(s) with a chemical or enzymatic agent between step (a) and step (b);
(f) wherein the disease is autosomal-dominant polycystic kidney disease, other chronic kidney diseases, kidney dysfunction, or preeclampsia;
(g) wherein said sample is selected from serum, plasma, whole blood, urine, CSF, ascites fluid, peritoneal fluid or other biological fluids;
(h) wherein the reference standard is a mean, an average, a numerical mean or range of numerical means, a numerical pattern, a ratio, a graphical pattern or a protein profile derived from the same Target or Targets in a reference subject or reference population; and
(i) wherein said diagnosis or detecting comprises early diagnosis of disease, determining the best clinical treatment, monitoring relapse after initial diagnosis and treatment, or predicting clinical outcome.

63-67. (canceled)


SEQUENCE LISTING (54 sequences)

SEQ ID NO: 1 - protein, 196 aa, Homo sapiens
MAAIRKKLVVVGDGACGKTCLLIVFSKDEFPEVYVPTVFENYVADIEVDGKQVELALWDTAGQEDYDRLRPLSYPDTDVILMCFSVDSPDSLENIPEKWVPEVKHFCPNVPIILVANKKDLRSDEHVRTELARMKQEPVRTDDGRAMAVRIQAYDYLECSAKTKEGVREVFETATRAALQKRYGSQNGCINCCKVL

SEQ ID NO: 2 - protein, 82 aa, Homo sapiens
VANKKDLRSDEHVRTELARMKQEPVRTDDGRAMAVRIQAYDYLECSAKTKEGVREVFETATRAALQKRYGSQNGCINCCKVL

SEQ ID NO: 3 - protein, 61 aa, Homo sapiens
KDLRSDEHVRTELARMKQEPVRTDDGRAMAVRIQAYDYLECSAKTKEGVREVFETATRAAL

SEQ ID NO: 4 - protein, 19 aa, Homo sapiens
RTDDGRAMAVRIQAYDYLE

SEQ ID NO: 5 - protein, 13 aa, Homo sapiens
DDGRAMAVRIQAY

SEQ ID NO: 6 - protein, 13 aa, Homo sapiens
RTDDGRAMAVRIQ

SEQ ID NO: 7 - protein, 11 aa, Homo sapiens
AVRIQAYDYLE

SEQ ID NO: 8 - protein, 12 aa, Homo sapiens
IQAYDYLECSAK

SEQ ID NO: 9 - protein, 12 aa, Homo sapiens
IEAYDYLECSAK

SEQ ID NO: 10 - protein, 8 aa, Homo sapiens
IQAYDYLE

SEQ ID NO: 11 - protein, 9 aa, Homo sapiens
RIQAYDYLE

SEQ ID NO: 12 - protein, 10 aa, Homo sapiens
VRIQAYDYLE

SEQ ID NO: 13 - protein, 9 aa, Homo sapiens
IQAYDYLEC

SEQ ID NO: 14 - protein, 10 aa, Homo sapiens
RIQAYDYLEC

SEQ ID NO: 15 - protein, 11 aa, Homo sapiens
VRIQAYDYLEC

SEQ ID NO: 16 - protein, 12 aa, Homo sapiens
AVRIQAYDYLEC

SEQ ID NO: 17 - protein, 10 aa, Homo sapiens
IQAYDYLECS

SEQ ID NO: 18 - protein, 11 aa, Homo sapiens
RIQAYDYLECS

SEQ ID NO: 19 - protein, 12 aa, Homo sapiens
VRIQAYDYLECS

SEQ ID NO: 20 - protein, 13 aa, Homo sapiens
AVRIQAYDYLECS

SEQ ID NO: 21 - protein, 11 aa, Homo sapiens
IQAYDYLECSA

SEQ ID NO: 22 - protein, 12 aa, Homo sapiens
RIQAYDYLECSA

SEQ ID NO: 23 - protein, 13 aa, Homo sapiens
VRIQAYDYLECSA

SEQ ID NO: 24 - protein, 14 aa, Homo sapiens
AVRIQAYDYLECSA

SEQ ID NO: 25 - protein, 13 aa, Homo sapiens
RIQAYDYLECSAK

SEQ ID NO: 26 - protein, 14 aa, Homo sapiens
VRIQAYDYLECSAK

SEQ ID NO: 27 - protein, 15 aa, Homo sapiens
AVRIQAYDYLECSAK

SEQ ID NO: 28 - protein, 8 aa, Artificial Sequence
  Synthetic amino acid sequence.
IEAYDYLE

SEQ ID NO: 29 - protein, 9 aa, Artificial Sequence
  Synthetic amino acid sequence.
RIEAYDYLE

SEQ ID NO: 30 - protein, 10 aa, Artificial Sequence
  Synthetic amino acid sequence.
VRIEAYDYLE

SEQ ID NO: 31 - protein, 11 aa, Artificial Sequence
  Synthetic amino acid sequence.
AVRIEAYDYLE

SEQ ID NO: 32 - protein, 9 aa, Artificial Sequence
  Synthetic amino acid sequence.
IEAYDYLEC

SEQ ID NO: 33 - protein, 10 aa, Artificial Sequence
  Synthetic amino acid sequence.
RIEAYDYLEC

SEQ ID NO: 34 - protein, 11 aa, Artificial Sequence
  Synthetic amino acid sequence.
VRIEAYDYLEC

SEQ ID NO: 35 - protein, 12 aa, Artificial Sequence
  Synthetic amino acid sequence.
AVRIEAYDYLEC

SEQ ID NO: 36 - protein, 10 aa, Artificial Sequence
  Synthetic amino acid sequence.
IEAYDYLECS

SEQ ID NO: 37 - protein, 11 aa, Artificial Sequence
  Synthetic amino acid sequence.
RIEAYDYLECS

SEQ ID NO: 38 - protein, 12 aa, Artificial Sequence
  Synthetic amino acid sequence.
VRIEAYDYLECS

SEQ ID NO: 39 - protein, 13 aa, Artificial Sequence
  Synthetic amino acid sequence.
AVRIEAYDYLECS

SEQ ID NO: 40 - protein, 11 aa, Artificial Sequence
  Synthetic amino acid sequence.
IEAYDYLECSA

SEQ ID NO: 41 - protein, 12 aa, Artificial Sequence
  Synthetic amino acid sequence.
RIEAYDYLECSA

SEQ ID NO: 42 - protein, 13 aa, Artificial Sequence
  Synthetic amino acid sequence.
VRIEAYDYLECSA

SEQ ID NO: 43 - protein, 14 aa, Artificial Sequence
  Synthetic amino acid sequence.
AVRIEAYDYLECSA

SEQ ID NO: 44 - protein, 13 aa, Artificial Sequence
  Synthetic amino acid sequence.
RIEAYDYLECSAK

SEQ ID NO: 45 - protein, 14 aa, Artificial Sequence
  Synthetic amino acid sequence.
VRIEAYDYLECSAK

SEQ ID NO: 46 - protein, 15 aa, Artificial Sequence
  Synthetic amino acid sequence.
AVRIEAYDYLECSAK

SEQ ID NO: 47 - DNA, 665 bp, Artificial Sequence
  light chain of 7F7
  misc_feature (76)..(121): CDR1
  misc_feature (169)..(187): CDR2
  misc_feature (286)..(340): CDR2
ccagttccgagctccagatgacccagactccactctccctgcctgtcagtcttggagatcaagcctccatctcttgcagatcaagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 48 - protein, 221 aa, Artificial Sequence
  light chain of 7F7
  MISC_FEATURE (26)..(41): CDR1
  MISC_FEATURE (57)..(63): CDR2
  MISC_FEATURE (96)..(114): CDR2
SSELQMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 49 - DNA, 1288 bp, Artificial Sequence
  heavy chain of 7F7
  misc_feature (70)..(82): CDR1
  misc_feature (127)..(175): CDR2
  misc_feature (301)..(343): CDR3
tgaggtgaagctggtggagactggggcttcagtgaagttgtcctgcaaggcttctggctacaccttcaccagctactatatgttctgggtgaagcagaggcctggacatggccttgagtggattggggggtttaatcctaccaatggtggtactgacttcaatgagaagttcaagagcaaggccaccctgactgtagacaagtcctccaccacagcctacatacaactcagcagcctgacatctgaggactctgcggtctattactgtacggatggtaacctctggggtcaaggaacctcggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 50 - protein, 429 aa, Artificial Sequence
  heavy chain of 7F7
  MISC_FEATURE (24)..(28): CDR1
  MISC_FEATURE (43)..(59): CDR2
  MISC_FEATURE (100)..(115): CDR2
EVKLVETGASVKLSCKASGYTFTSYYMFWVKQRPGHGLEWIGGFNPTNGGTDFNEKFKSKATLTVDKSSTTAYIQLSSLTSEDSAVYYCTDGNLWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 51 - DNA, 647 bp, Artificial Sequence
  light chain of 9G5
  misc_feature (76)..(103): CDR1
  misc_feature (151)..(169): CDR2
  misc_feature (268)..(325): CDR3
ccagttccgagctccagatgacccagactccagcaatcatgtctgcatctccaggggagaaggtcaccatgacctgcagtgccagctcaagtgtaagttacatgcactggtaccagcagaagccaggatcctcgcccaaaccctggatttatgacacatccaacctggcttctggattccctgctcgcttcagtggcagtgggtctgggacctcttactctctcataatcagcagcatggaggctgaagatgctgccacttattactgccatcagcggagtagttacccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 52 - protein, 215 aa, Artificial Sequence
  light chain of 9G5
  MISC_FEATURE (26)..(35): CDR1
  MISC_FEATURE (51)..(57): CDR2
  MISC_FEATURE (90)..(109): CDR3
SSELQMTQTPAIMSASPGEKVTMTCSASSSVSYMHWYQQKPGSSPKPWIYDTSNLASGFPARFSGSGSGTSYSLIISSMEAEDAATYYCHQRSSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - DNA, 1359 bp, Artificial Sequence
  heavy chain of 9G5
  misc_feature (91)..(103): CDR1
  misc_feature (148)..(202): CDR2
  misc_feature (301)..(352): CDR3
gaggtgaagctggtggagacwggtggaggattggtgcagcctaaagggtcattgaaactctcatgtgcagcctctggattcaacttcaatacctacgccatgaactgggtccgccaggctccaggaaagggtttggaatgggttgctcgcataagaagtaaaagtaataattatgcaacatattatgccgattcagtgaaagacagattcaccatctccagagatgattcagaaaacatgctctatctgcaaatgaacaacttgaaaactgaggacacagccatttattactgtgtgagagggggtggtaaccttgactactggggccaaggcaccactctcacagtctcctcagccaaaacaacagccccatcggtctatccactggcccctgtgtgtggaggtacaactggctcctcggtgactctaggatgcctggtcaagggttatttccctgagccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccttctcccggactccgggtaaa

SEQ ID NO: 54 - protein, 453 aa, Artificial Sequence
  heavy chain of 9G5
  MISC_FEATURE (31)..(35): CDR1
  MISC_FEATURE (50)..(68): CDR2
  MISC_FEATURE (101)..(118): CDR3
EVKLVETGGGLVQPKGSLKLSCAASGFNFNTYAMNWVRQAPGKGLEWVARIRSKSNNYATYYADSVKDRFTISRDDSENMLYLQMNNLKTEDTAIYYCVRGGGNLDYWGQGTTLTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTFSRTPGK